(19) United States
(12) Patent Application Publication
(10) Pub. No.: US 2021/0378730 A1
(43) Pub. Date: Dec. 9, 2021

(54) REMOVABLE INTEGRATED ACTUATOR ASSEMBLY FOR ELECTROSURGICAL FORCEPS

(71) Applicant: BIPAD, INC., Point Lookout, NY (US)

(72) Inventor: Louis G. Cornacchia, III, Point Lookout, NY (US)

(73) Assignee: BIPAD, INC., Point Lookout, NY (US)

(21) Appl. No.: 17/421,434

(22) PCT Filed: Nov. 27, 2019
(86) PCT No.: PCT/US19/63550

§ 371 Date: Jul. 8, 2021

Related U.S. Application Data
(60) Provisional application No. 62/795,049, filed on Jan. 22, 2019.

(51) Int. Cl. A61B 18/14

(52) U.S. Cl. CPC A61B 18/1442; A61B 2018/126

(57) ABSTRACT

An actuator assembly connects to a tool plug at a proximal end of a conventional bipolar electrosurgical forceps having electrodes at a distal end for applying electrical current to tissue. A switch body with an integral power cord includes a plug mount that accepts the tool plug and a switch for connecting the tool plug to an electrical generating apparatus when the switch is closed. An actuator body mounted to the switch body includes a pivotally mounted switch actuating member to which an actuator lever arm is adjustably mounted. The switch body and actuator body are configured so that when the tool is held in the user's hand with the plug mount secured to the tool plug, the actuator lever arm can be positioned for movement by a finger of the user's hand to move the switch actuating member and close the switch.

### CROSS-REFERENCE TO RELATED APPLICATION

This application claims the benefit of U.S. provisional application No. 62/795,049, filed Jan. 22, 2019, the entire contents of which are incorporated herein by reference.

### BACKGROUND OF THE INVENTION

### Field of the Invention

The present invention relates to an actuator assembly for a bipolar forceps, and more particularly, to an integrated actuator assembly mounted to a bipolar forceps for facilitating multi-mode, one-hand operation thereof.

### Description of Related Art

Modern electrosurgery dates from the discovery about 100 years ago that applying electrical current at radio frequencies to living tissue will coagulate blood. Passing an RF electrical current through tissue heats and cauterizes it, reducing blood loss and thereby promoting better patient outcomes. Electrosurgery has become widespread today in many surgical contexts, and the basic principles underlying electrosurgery are well known. However, apparatus for performing electrosurgery has taken many forms, none of which has proven entirely satisfactory.

Basic components of an electrosurgical arrangement of the type with which the present disclosure is concerned are an electrosurgical tool and an electrical generating apparatus. The electrosurgical tool typically comprises a forceps with two insulated tines, each of which has an exposed electrode at a distal region. The tines extend along a generally longitudinal axis to a proximal region with a tool plug that is electrically connected to the tool electrodes by conductors inside the tines. A power cord removably connects the tool plug to the electrical generating apparatus for applying electrical current to the electrodes. The tines have a handle portion at the forceps' proximal region whereby a user holding the forceps can squeeze the tines together to capture tissue between them. Introducing current to the tool plug from the electrical generating apparatus via the power cord heats and cauterizes tissue between the electrodes.

In an arrangement widely used today the electrical generating apparatus is selectively actuated by a foot pedal. When the forceps have been manipulated to capture the desired tissue between the forceps' electrodes, the medical professional performing the procedure, or an assistant, steps on the foot pedal to close a switch in the electrical generating apparatus and, via the power cord, introduce current to the tool plug and thus to the electrodes. Typically, the person performing the procedure locates the pedal by “feel.” In a procedure in which the forceps' electrodes must be positioned with precision, it is difficult both to concentrate on the surgical field and to look at the floor to locate the pedal. The applicant's U.S. Pat. No. 9,433,460 describes some of the shortcomings of foot pedal systems, such as the location of the pedal sometimes not being aligned with the user's foot, or requiring that the user grope for the pedal or contort his or her body position to depress the pedal, thus posing significant risk and possibly causing delays that compromise the procedure. Having someone other than the person performing the procedure move the pedal, such as a surgeon's assistant, can also cause delay. Further, if the surgeon has to move to a different location during the procedure, he or she may not be able to readily locate the pedal without looking away from the patient. (At times this description will refer to “the surgeon” performing a procedure. It will be understood that this includes users other than those who would normally be deemed surgeons in strict medical parlance.)

One approach for addressing this issue is to place a switch at a location where it can be actuated by the user's hand holding the forceps. U.S. Pat. No. 5,116,333 to Beane (assigned to Kirwan Surgical Products, Inc.) represents an early example of this approach. Beane's handswitch adapter is intended to permit a surgeon to use the same hand to manipulate a bipolar forceps at a surgical site and actuate a switch carried by the forceps. The adapter, which includes the switch, is a unitary structure separate from the forceps and the power cord. It includes a fixed-length extension that has one end secured to an adapter base and that extends along the forceps' longitudinal axis. A reed switch mounted at the other end of the extension is closed when the user presses on it with a fingertip. This construction has a number of drawbacks. It will be appreciated from Beane's FIG. 1 that requiring the user to press on the reed switch located at the tip end of the extension may prove awkward for some users and could risk inadvertently moving the forceps and compromising the procedure. Moreover, the extension lies in a plane between the forceps' tines, making it even more awkward for the user to hold the forceps steady while reaching toward the reed switch. In addition, the construction of the adapter makes it inconvenient to alternate between hand operation and foot-pedal operation with the adapter in place, or to use the forceps without the adapter, all of which may be preferred by a given surgeon at different times during a procedure. Beane does not describe a way of converting between these modes of operation without unplugging the forceps from the power cord, removing the adapter from the forceps, and plugging the forceps back into the power cord. Other drawbacks include the difficulty of sterilizing the adapter without damaging the fragile reed switch, and the cost of the reed switch in the first place.

U.S. Pat. No. 9,433,460 avoids many of Beane's shortcomings. It interposes between the forceps and power cord an actuating component with a push-button switch. On one side the actuating component has sockets that mimic the sockets on a conventional power cord plug and on the other side it has prongs that mimic the prongs on a conventional tool plug of a bipolar forceps. The actuating component has a lever arm that the user presses with a finger of the hand holding the forceps tines to move the lever arm against the push button on the switch to close a circuit and introduce current to the tool plug from the electrical generating apparatus via the power cord. This configuration places the lever arm at a location proximate to the natural location of the user's finger when he or she is holding the forceps with the thumb on one tine and the index or middle finger on the other. See, for example, FIGS. 16 and 17 of the applicant's Pub. No. US 2018/0055558, and FIG. 9 herein. U.S. Pat. No. 9,433,460 permits the surgeon to use a foot pedal to introduce current to the forceps when the actuating component is attached between the tool plug and the power cord plug. However, if the surgeon wants to use the forceps without the actuating arm in the way, he or she must still disconnect the tool and the power cord from the actuating component and reconnect them together directly.

Pub. No. US 2018/0055558 includes some of the basic configurational features of the actuating arrangement in U.S. Pat. No. 9,433,460, in that it includes an actuator assembly with a lever arm that presses on a push-button switch when the user pushes on the lever arm with a finger of the hand holding the forceps. It improves on the arrangement in U.S. Pat. No. 9,433,460 by making the power cord and actuator assembly a unitary structure so that it can be immediately connected in place on the tool plug ready for use. Another feature of the actuator assembly in the '558 publication is the ergonomic shape of the lever arm, which is designed so that it more closely matches the position and contour of a user's finger when the forceps is in use. While integrating the actuator assembly and power cord makes it quicker and easier to convert the forceps to hand actuation, it does not readily allow for using the forceps without the actuator arm. That requires disconnecting the actuator assembly from the tool plug and the electrical generating apparatus and replacing it with a conventional power cord. In addition, converting between right- and left-hand configurations using the ergonomically curved lever arm described in Pub. No. US 2018/0055558 requires different lever arms, thus increasing the number of small parts that must be furnished with each unit. An additional feature that could affect the utility of the configuration is the fixed distance by which the lever arm extends along the tines, which doesn't account for the fact that different users have different size hands, or may prefer different-length lever arms for different procedures.

What is needed is an actuator assembly that permits a surgeon to control the provision of electrical current to a bipolar forceps with the same hand gripping the forceps. The actuator will preferably have a construction that places an actuating component such as a lever arm where a finger of the surgeon's hand is naturally located during use of the forceps. It should also permit removal of the lever arm so that the supply of electrical current can be controlled solely by a foot pedal in the conventional manner, without requiring the power cord to be separated from the tool, and preferably be easily converted between left- and right-hand operation.

### BRIEF DESCRIPTION OF THE DRAWINGS

The detailed description that follows below will be better understood when taken in conjunction with the accompanying drawings, in which like numerals and letters refer to like features throughout. The following is a brief identification of the drawing figures used in the detailed description.

FIG. 1 is a perspective view of a conventional bipolar electrosurgical forceps to which an actuator assembly according to an embodiment of the invention is mounted, depicting the manner in which the forceps connects to an electrical generating apparatus via the actuator assembly.

FIG. 2 is an exploded perspective view of the bipolar forceps and actuator assembly depicted in FIG. 1 showing further details of the actuator assembly's switch body with a unitary power cord, and a separate actuator body and separate actuator lever arm.

FIG. 3 is an exploded perspective view of the embodiment depicted in FIG. 1 from another angle illustrating the constructional relationship between the various parts of the actuator assembly and the forceps.

FIG. 4 is an exploded perspective view showing parts of the actuator assembly and how it is removably mounted to the switch body.

FIG. 5 is a detailed perspective view of the switch actuating member of the present embodiment.

FIG. 6 is a sectional view taken along lines 6-6 in FIG. 5.

FIG. 7 is a side view of the actuator lever arm of the present embodiment.

FIG. 8 is a sectional view taken along lines 8-8 in FIG. 7.

FIG. 9 illustrates the actuator lever arm in a first configuration oriented for right-handed operation in a first mode via the user's index finger.

FIG. 10 illustrates the actuator lever arm in a second configuration in which it is bent slightly upward as compared to the first configuration shown in FIG. 9.

FIG. 11 illustrates right-handed operation of the actuator assembly in the configuration shown in FIG. 10 in a second mode via the tip of the user's index finger.

FIG. 12 illustrates the actuator lever arm in a third configuration in which it is bent downward as compared to the first orientation shown in FIG. 9 for right-handed by the user's third finger in a third mode of operation.

FIG. 13 is a perspective view of the bipolar forceps mounted to the actuator assembly of FIG. 1 for left-handed operation.

One skilled in the art will readily understand that the drawings are not strictly to scale and are generally schematic in nature, but nevertheless will find them sufficient, when taken with the detailed description that follows, to make and use the devices and practice the methods described herein.

### SUMMARY OF THE INVENTION

It is one object of the present invention to provide an actuator assembly that can be used with a conventional bipolar electrosurgical forceps and can assume a variety of different configurations to give a surgeon maximum flexibility in the manner in which a procedure using the forceps is performed.

A construction featured in one embodiment of the invention comprises a three-component actuator assembly that in various combinations enables a degree of operational flexibility heretofore missing from handheld actuators for electrosurgical forceps. This actuator assembly includes a switch body with a power cord for introducing electrical current to the forceps from a conventional electrical generator. The switch body mounts to the forceps tool plug in a like manner to known power cord plugs. The actuator assembly further includes an actuator body mounted on the switch body and an actuator lever arm movable by a user's finger while holding the forceps. Movement of the lever arm actuates a switch in the switch body to introduce electric current to the forceps.

In one variation the actuator assembly includes three separate components: a switch body integrated with the power cord, an actuator body removably mountable to the switch body, and an actuator lever arm adjustably mounted to the actuator body. This construction permits a surgeon to use an actuator assembly including all three components for one hand operation of the forceps, while permitting removal of the actuator body/lever arm subassembly from the switch body without unplugging the switch body from the forceps tool plug. This allows the surgeon to readily convert to foot pedal operation alone if it would facilitate a particular part of a procedure (for example, if the lever arm obstructs the surgical field). In another variation, the lever arm can be removed from the actuator body while leaving the latter mounted to the switch body.

Another aspect of the invention resides in the configuration and mounting of the actuator lever arm. The actuator lever arm is carried by a switch actuating member mounted for movement relative to the actuator body. When the user moves the lever arm, the switch actuating member closes the switch to introduce electrical current to the forceps. The actuator body and switch actuating member are configured to place the lever arm in position for movement by a user's finger when the user grasps the forceps. The lever arm includes a shaft slidingly received in the switch actuating member and an enlarged distal contact portion shaped so the user can readily locate and operate it by feel during a procedure.

Certain aspects of the actuator lever arm in various embodiments are particularly advantageous. The lever arm shaft can be made plastically deformable to permit each user to position the contact portion relative to the forceps according to his or her preference. The contact portion is preferably curved generally convex-outward relative to the forceps' tines where the user grips them. This provides tactile feedback that lets the surgeon know immediately if his or her finger is properly positioned on the contact portion. In addition the contact portion surface can be contoured to more positive contact in the presence of fluids during a surgical procedure. Alternately, or additionally, the contact portion can have cutouts that provide further tactile feedback allowing the surgeon to properly position his or her finger on the contact portion for optimum results.

In yet another embodiment at least the switch body and actuator body comprise a unitary structure that can be connected to and disconnected intact from the forceps tool plug. This will simplify manufacture and facilitate use of the actuator assembly by constituting it of fewer individual parts. In one form of this embodiment the lever arm is removably mounted to the actuator body so that it can be removed to provide an unobstructed view of the surgical field during a procedure without removing the integrated switch body and actuator body subassembly. In still another alternate embodiment the forceps, switch body, and power cord comprise an integral disposable unit that can be discarded after a single use to avoid sterilization issues.

These and other aspects and features of the invention and embodiments thereof will be covered in more detail as this description proceeds. A Summary of the invention has been provided here solely to introduce in a simplified form a selection of concepts that are described in detail below and is not intended necessarily to identify key or essential features of the subject claimed herein.

### DETAILED DESCRIPTION OF PREFERRED EMBODIMENTS

Embodiments are described more fully below in sufficient detail to enable those skilled in the art to use the described medical instruments and methods. However, embodiments may be implemented in many different forms and should not be construed as being limited to the embodiments set forth herein. The following detailed description is, therefore, not to be taken in a limiting sense. This description is intended to provide specific examples of particular embodiments illustrating various ways of implementing the claimed subject matter. It is written to take into account the level of knowledge of one of ordinary skill in the art to which the claimed subject matter pertains. Accordingly, certain details may be omitted as being unnecessary for enabling such a person to realize the embodiments described herein. In addition, spatially relative terms such as “upward,” “downward,” “top,” “bottom,” “right,” “left,” “under,” “over,” “proximal,” “distal,” etc., may be used herein for convenience, but they in no way limit the structure or procedure described, unless the context indicates otherwise. Similar considerations apply to the term “about,” which is sometimes used herein to indicate that the nominal value of a parameter can vary a certain amount as long as it produces the intended effect or result.

In addition, terms used throughout are meant to have the ordinary and customary meaning that would be ascribed to them by one of ordinary skill in the art. However, some of the terms used in the description herein will be explicitly defined and that definition is meant to apply throughout. For example, the term “substantially” is sometimes used to indicate a degree of similarity of one item, such as a property, structural feature, or parameter, to another. This means that the items are sufficiently similar to achieve the purpose ascribed to them in the context of the description accompanying the use of the term. Exact equivalence of many items discussed herein is not possible because of factors such as engineering tolerances and normal variations in operating conditions, but such deviations from an exact identity still fall within the meaning herein of being “substantially” the same. Likewise, omission of the term “substantially” when equating two such items does not imply that they are identical unless the context suggests otherwise.

When elements are referred to as being “connected” or “coupled,” the elements can be directly connected or coupled together or one or more intervening elements may also be present. In contrast, when elements are referred to as being “directly connected” or “directly coupled,” there are no intervening elements present.

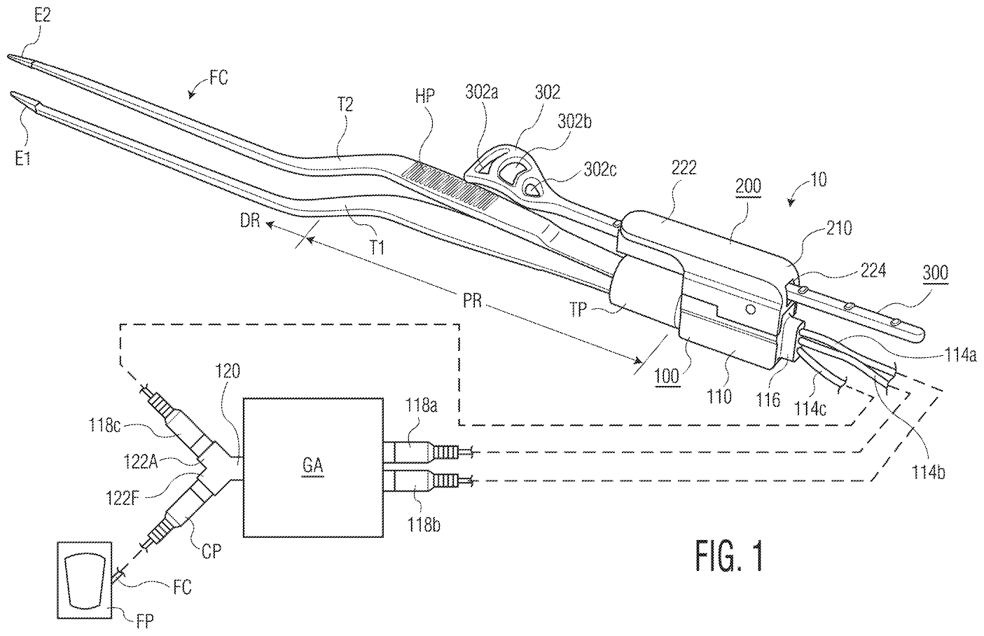

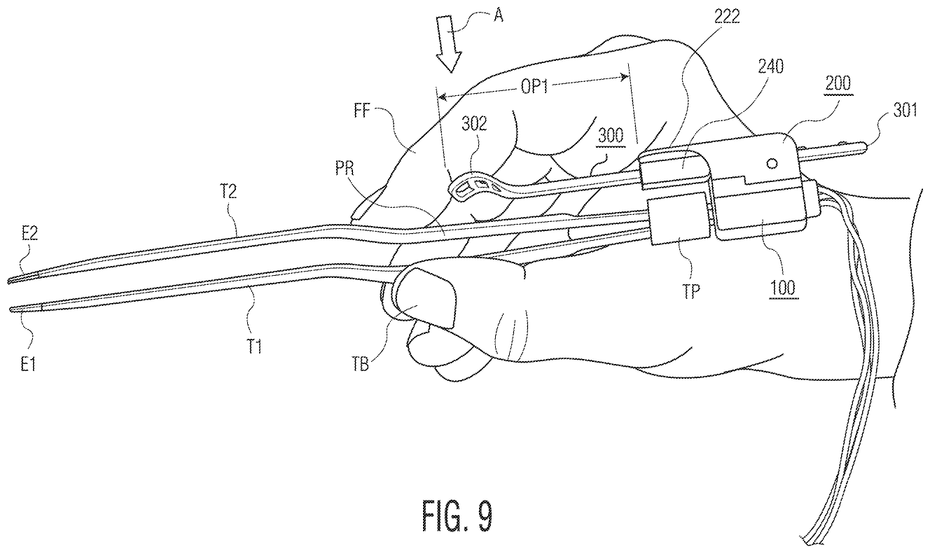

FIGS. 1-4 illustrate the overall configuration of the manner in which the particular embodiment of the novel actuator assembly described herein cooperates with a conventional prior art bipolar forceps and electrical generating apparatus to facilitate the accurate and precise application of electrical current at a desired location. FIG. 1 is a perspective view showing a prior art bipolar electrosurgical tool in the form of a forceps FC extending generally between a proximal region PR and a distal region DR. The proximal region ends at a tool plug TP to which a first, left tine T1 and second, right tine T2 are attached. The tines terminate at distal electrodes E1 and E2, respectively, that are electrically connected to the tool plug through conductors disposed internally of the insulating tines. A tool longitudinal axis extends generally between the tool plug TP and the electrodes E1 and E2. For right-handed operation, the user grasps the forceps FC with one hand, placing his or her thumb on the first tine T1 and a finger, usually the index or middle finger, on the second tine T2, in a manner described in more detail below in connection with FIGS. 9-12. In a typical prior art arrangement a power cord from an electrical generating apparatus GA terminates in a connector with sockets that accept prongs on the tool plug. (Atypical prior art set up of this type is shown in U.S. Pat. No. 9,433,460.) The surgeon captures the target tissue between the electrodes E1 and E2 and depresses a foot pedal FP that completes an electrical circuit through the tissue. This type of setup has been in widespread use for many years, and surgeons are comfortable using it in delicate medical procedures where precision placement of the electrodes is critical. Accordingly, configurational changes that change the “feel” of this basic device or alter the manner in which it is manipulated into position during a procedure will meet resistance from surgeons who employ it extensively in their practices. By the same token, an alternative to exclusive foot pedal operation would be desirable for reasons already discussed.

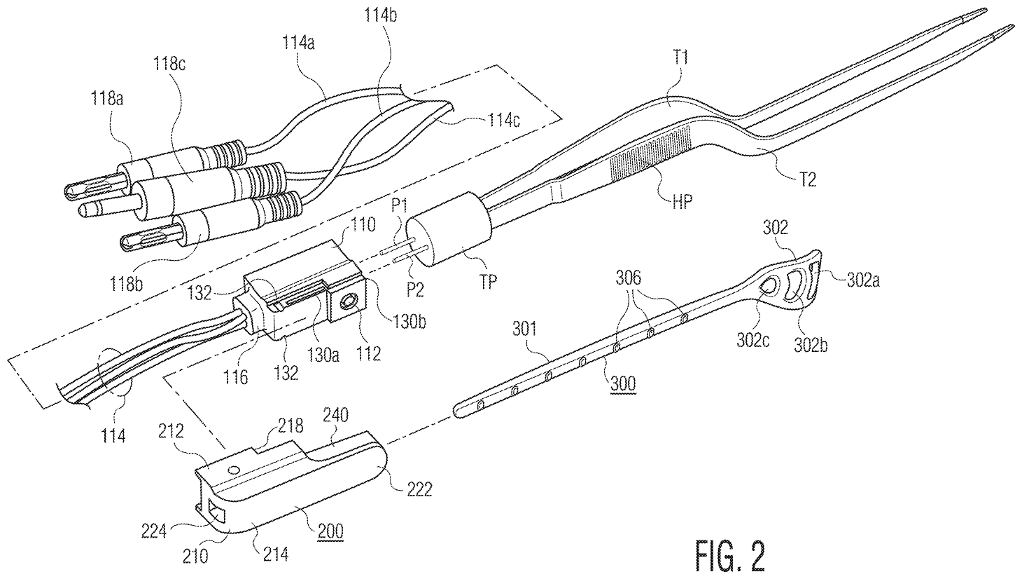

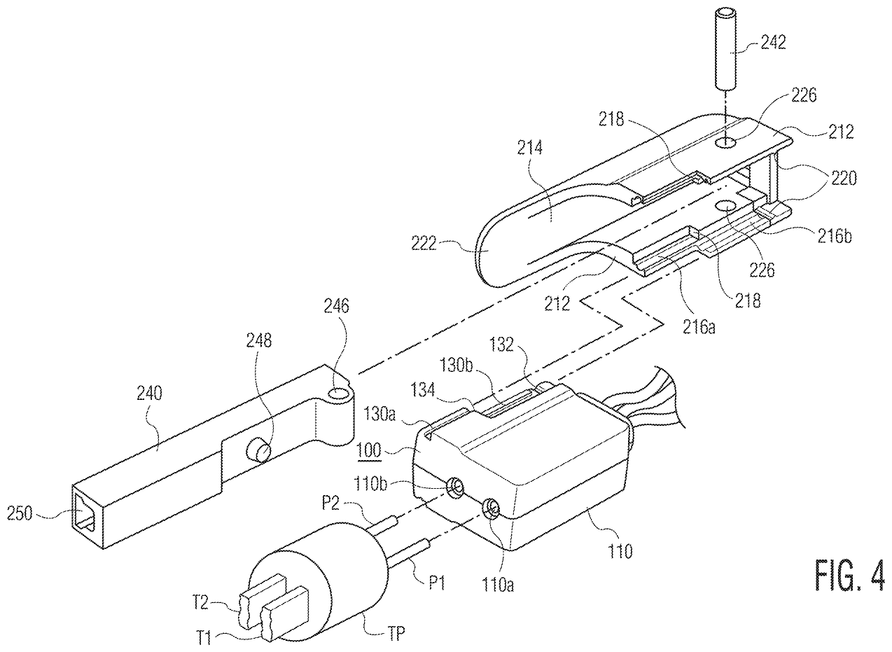

To that end, the present disclosure describes a configuration that enables actuation of the electrodes E1 and E2 by a user without requiring the operation of a foot pedal, while permitting the forceps to be held and manipulated into position with familiar techniques used with the old set up. As shown in FIGS. 1-4, this new configuration uses a novel actuator assembly 10 in place of the conventional prior art power cord and tool plug connector previously used to conduct current from the electrical generating apparatus. A first principal component of the actuator assembly 10 is a switch body 100 that includes a plug mount 110 with sockets 110a and 110b (see FIGS. 2-4 and 3) for accepting mating prongs P1 and P2 on the tool plug TP. An important feature of the present embodiment is the ability to mount the tool on the plug mount with the prongs P1 and P2 in respective plug mount sockets 110a and 100b (see FIG. 4), which enables right-hand operation as depicted in FIGS. 1-3, or with the prongs P1 and P2 in respective plug mount sockets 110band 100a for left-hand operation. This feature is described in more detail further below in connection with FIGS. 9-13. The plug mount 110 has ridges 130a and 130b. A female detent 132 is provided at the proximate end of the ridge 130a. The ridges 130a and 130b are separated by a shoulder 134. The purpose of these features is explained further below in more detail in connection with FIG. 4.

The plug mount 110 includes a switch that comprises switch contacts within the plug mount and a spring-biased push-button actuator 112 for selectively placing the switch contacts in the plug mount in an open position in which they are not in electrical contact and a closed position in which current is conducted between the contacts. The switch is in an electrical circuit between a power cord 114 and the sockets 110a and 110b, whereby depressing the push-button actuator 112 against its spring bias electrically connects the electrical generating apparatus GA to the tool plug prongs P1 and P2 (and thus to the electrodes E1 and E2). An important feature of the actuator assembly 10 is its ability to be directly substituted for a conventional power cord that connects at one end to a conventional electrical generating apparatus, while still enabling at the discretion of the user either foot pedal operation or operation using the actuator assembly as described below. To that end, the power cord 114 includes three leads 114a, 114b, and 114c integrated with the plug mount 110 in a suitable manner, such as securing them in place via a molded collar 116 that captures the leads and holds them securely in place to from an integrated switch body/power cord assembly. The leads 114a and 114b comprise power leads that terminate at respective power plugs 118a and 118b that plug into the electrical generating apparatus's power outlets (not shown) in the same manner as a conventional power cord. The lead 114c comprises a control cord that terminates at a control plug 118c that is connected to the electrical generating apparatus GA.

As noted, another important feature of the actuator assembly 10 is that it can be used with conventional electrical generating apparatus and any of various conventional foot pedal actuators FP. A typical foot pedal actuator will include the foot pedal itself and a foot pedal control cord FC with a pedal control plug CP that plugs into a control socket on the apparatus GA. Electrical generating apparatus is typically available in either of two types. The apparatus GA in FIG. 1 represents one type, an example of which is the Codman® Malis® CDC® III or IV bipolar electrosurgical generator. For use with this type of generating apparatus, the actuator assembly 10 will typically be provided with a Y-connector 120 having prongs on the straight leg of the Y that plug into the control socket on the apparatus GA, and sockets on respective legs 122A and 122F of the Y. The socket 122A accepts the control plug 118c from the actuator assembly 10 and the socket 122F accepts the foot pedal control plug CP. The actuator control input and the foot pedal control input are essentially connected by the Y-connector in a parallel electrical circuit with the generating apparatus. (Preferably, the control sockets 112A and 122F are identical, and the control plug 118c is the same as the pedal control plug CP, so that the user can insert either plug into either socket.) When the foot pedal is depressed it closes a circuit that provides current to the prongs P1 and P2 of the tool plug via the power cords 114a and 114b in the conventional manner. When the switch of the actuator assembly is closed, it completes a circuit that provides current to the tool prongs P1 and P2 independent of the foot pedal control input. For use with another type of conventional electrical generating apparatus, exemplified by the Valleylab™ Force FX™ generator sold by Medtronic plc, the power leads 114a and 114b and the control cord 114c can terminate at a specially constructed, unitary three-prong plug, two of which carry electrical current to the forceps in response to a control input on the third.

FIG. 4 is an exploded view of the actuator assembly 10 that depicts constructional details of an actuator body 200 that comprises a second principal component of the actuator assembly 10. The actuator body 200 comprises an actuator housing 210 that is preferably molded in one piece with side walls 212 depending from a top wall 214. Grooves 216a and 216b are molded into the internal surfaces of the depending side walls for accepting the ridges 130a and 130b to provide connecting structure that permits a user to slide the actuator housing onto and off of the plug mount 110 as indicated by the dot-dash lines in FIGS. 2-4. A shoulder 218 separates the grooves 216a and 216b and cooperates with the shoulder 134 on the plug mount 110 to form a stop that positions the actuator housing 210 on the plug mount 110 with their proximal and distal ends flush, as shown in the assembled view in FIG. 1. A raised male detent 220 proximate to the end of each groove 216a is accepted into the cooperating female detents 132 on the plug mount 110 to provide a positive “click” indication to the user that the actuator housing 210 is properly seated on the plug mount 110 and to prevent inadvertent separation of these parts during a procedure. Other salient features of the actuator housing 210, discussed in more detail below, include a projecting hood 222 that extends the top wall 214 in a longitudinal direction, an opening 224 through the housing's proximal wall, and aligned holes 226 through the housing's side walls 212.

The connecting structure for removably mounting the actuator body can take other forms besides the exact configuration depicted in the drawings. For example, in one alternate construction the connecting structure could comprise ridges molded on the actuator housing with the cooperating grooves provided in the plug mount. In another construction the actuator housing side walls could be made sufficiently flexible to permit the actuator housing to snap onto the tool plug from the side (as seen in FIG. 4). Those skilled in the art will recognize many other constructions that can accomplish the purpose of removably securing the actuator body to the switch body.

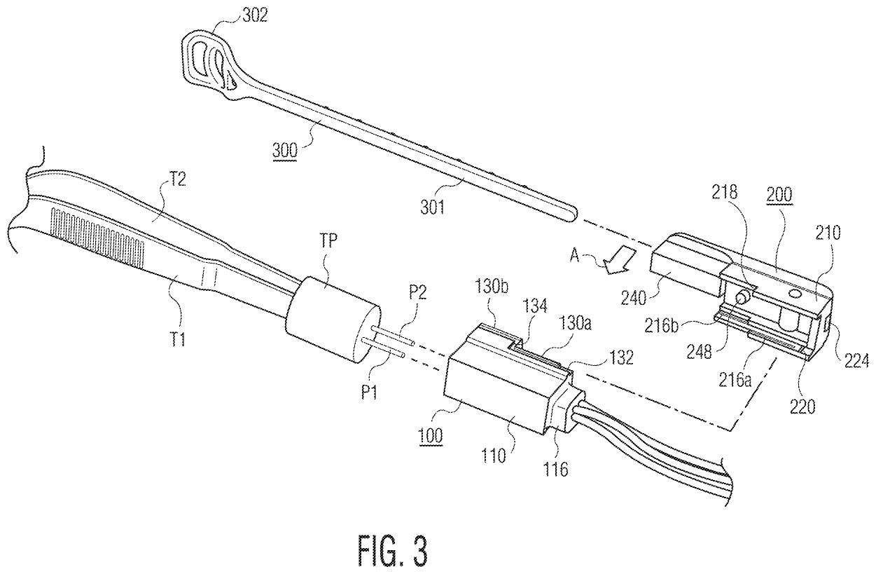

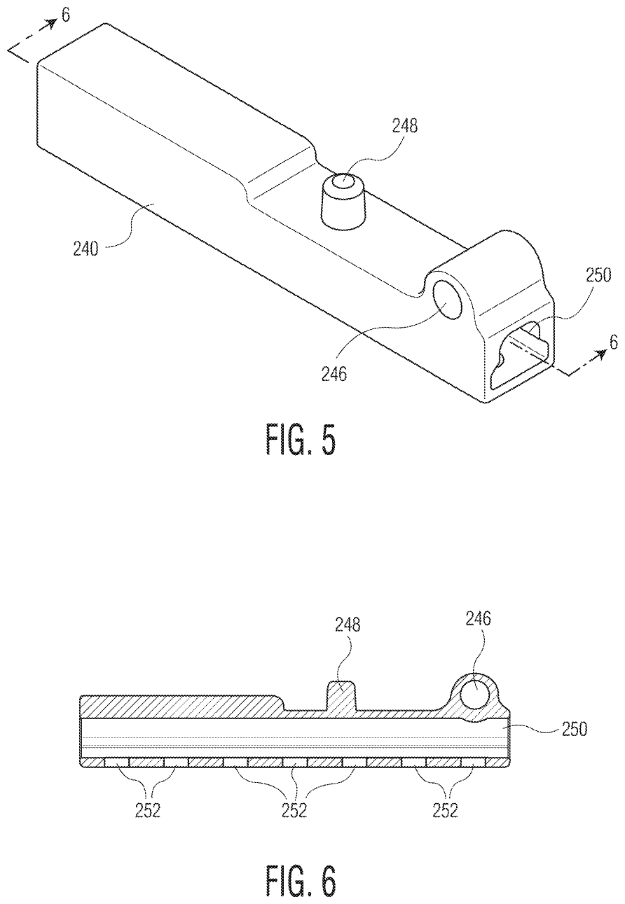

The actuator body 200 shown in FIG. 4 also comprises a one-piece, molded internal pivot arm 240, further details of which are depicted in FIGS. 5 and 6. The pivot arm and actuator housing are assembled into a unitary structure via a pivot pin 242, the ends of which are firmly and permanently secured to the holes 226 in the actuator housing side walls 212, and which passes through a clearance hole 246 at a proximal end of the pivot arm 240. The pivot pin 242 and the clearance hole 246 together define a hinge point about which the pivot arm 240 rotates relative to the actuator housing 210. The pivot arm 240 acts as a switch actuating member by rotation about the hinge point to bring an actuating button 248 on the pivot arm into contact with the push-button actuator 112 of the switch. It will be appreciated that the shoulders 134 on the plug mount 110 cooperate to place the actuating button 248 into juxtaposition with the switch's push-button actuator whereby rotation of the pivot arm 240 in the direction of the arrow A (see FIGS. 3 and 9) will depress the push button and close the switch. The pivot arm also has a longitudinal through-passage 250 and detent receptacles 252 along the wall opposite the wall carrying the actuating button 248.

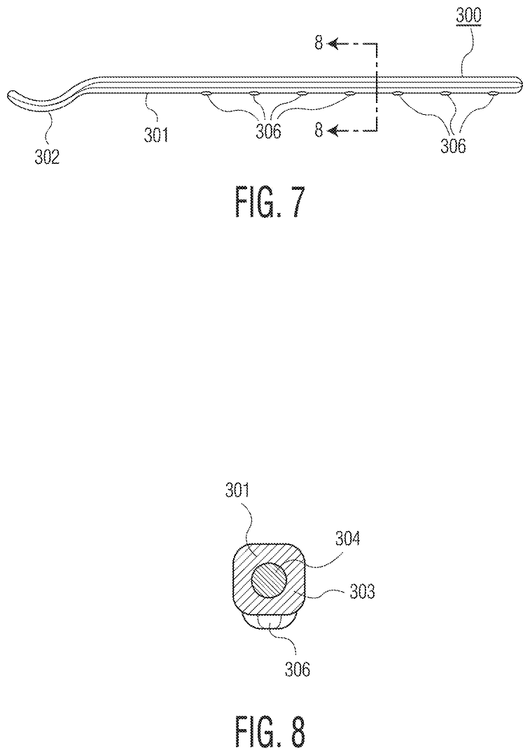

FIGS. 2 and 3, taken with FIGS. 7 and 8, depict constructional details of an actuator lever arm 300 that comprises a third principal component of the actuator assembly 10. The actuator lever arm comprises a shaft 301 terminating at one end at a contact portion 302. The shaft 301 comprises a sheath 303 molded around a core 304 of a stainless steel alloy capable of being deformed plastically. The lever arm 300 fits slidlingly within the longitudinal passage 250 of the pivot arm 240, as shown in FIG. 1 and indicated by dot-dash lines in FIGS. 2 and 3. Detent protrusions 306 molded on one side of the lever arm shaft cooperate with the detent receptacles 252 of the pivot arm to hold the lever arm in the position desired by the user. In a preferred embodiment the spacing between the detent receptacles is about 3-4 mm, which permits the contact portion 302 to be positioned relative to the forceps' tines to a sufficiently fine degree to allow operation by most users in accordance with the discussion below in connection with FIGS. 9-12. The space between each two protrusions 306 is twice as far as the space between the detent receptacles to reduce the force needed to slide the shaft 301 within the passage 250. The manner in which the detent protrusions and receptacles position the contact portion 302 relative to defined handle surfaces HP found on most conventional forceps can be seen in FIG. 1, and also in FIGS. 9-12 showing the actuator assembly in use. However, the term “handle portion” as used in the present disclosure and the claims that follow refers to any location on the forceps' tines where the user grips them for manipulation during a procedure and is not limited to the handle surfaces HP. The detent protrusions and detent receptacles cooperate to form positioning means for releasably holding the actuator lever arm in a plurality of positions relative to the pivot arm, as well as permitting the lever arm to be removed from the pivot arm completely. The positioning means can assume a variety of constructions for achieving the same result. For example, the protrusions can be on the inside surface of passage 250 and the receptacles can be in the form of dimples in the shaft 301. In another alternate construction the shaft can be held in position by frictional engagement with the passage walls. Another construction could use mating threads on the shaft 301 and on the inside of the passage 250. All of those various forms and their equivalents that perform the same functions of permitting adjustment of the position of the lever arm and/or its removal from the pivot arm are included within the meaning of “positioning means” as used herein. In a still further embodiment the actuator lever arm can be permanently attached to the pivot arm either in a fixed position relative to the pivot arm or in a manner that permits its position to be adjusted. One way of realizing the latter arrangement would be to include a knob (not shown) on the proximal end of the lever arm shaft to prevent it from being withdrawn from the passage 250 in the pivot arm.

The lever arm 300 terminates in the enlarged contact portion 302, which is specifically designed to facilitate operation by a user's finger. The plastically deformable steel core of the lever arm shaft 301 permits it to be bent into various shapes to place the enlarged contact portion 302 at a particular configuration depending on a user's preference, a feature that is described in more detail in the next paragraphs explaining the actuator assembly 10 in operation. The ability of the lever arm to be bent into a desired shape and adjusted to extend from the pivot arm by a distance according to a user's preference provides a level of versatility missing from prior art hand-actuated bipolar forceps—including the ability to remove the lever arm and use foot pedal actuation exclusively—which will be apparent from the following description of just some of the different methods of using the actuator assembly described herein.

FIGS. 9-13 describe how the novel actuator assembly with the features just described gives a user a wide variety of options for using a conventional bipolar forceps, and increases the convenience of changing between different modes of operation during a surgical procedure. A first mode of operation will be described by assuming that the actuator body housing 210 is mounted on the plug mount 110 of the switch body 100, with the lever arm 300 in place in the pivot arm 240 in the configuration shown in FIG. 1. The lever arm 300 in this mode is straight and extends from the pivot arm alongside the forceps' handle portion.

As shown in FIG. 9, the user grasps the forceps with the thumb TB and first finger FF on opposing handle portions. Before the procedure the user typically will have adjusted the distance OP1 by which the lever arm extends from the pivot arm so that the contact portion 302 is juxtaposed with the inside of his or her finger FF between the second and third knuckles. This places the contact portion 302 at a location proximate to the forceps' handle portion (see FIG. 1) that permits the user to move the lever arm in the direction of the arrow A by slightly straightening the finger FF to rotate the pivot arm about the hinge point provided by the pivot pin 242. This causes the actuating button 248 on the pivot arm 240 to depress the push-button switch actuator 112, which closes the switch and introduces current to the electrodes E1 and E2. The enlarged contact portion is curved convex-outward relative to the forceps (see FIG. 7), and thus conforms generally to the inside surface of the users' finger in FIG. 9 where it rests on the contact portion. The enlarged contact portion provides surface-to-surface contact with the user's finger to improve the user's ability to tactilely position his or her finger on the enlarged portion and thus more precisely control the application of electrical current during a procedure. Optionally, the surface of the enlarged portion contacted by the user's finger has contours to provide additional tactile input to the user. In the embodiment shown in the drawings the contours comprise three cutouts 302a, 302b, and 302c molded into the lever arm. However, other configurations for enhancing the users' ability to tactilely locate the lever arm are possible. For example, the cutouts could instead be depressions molded into the lever arm.

FIG. 9 also illustrates another feature of a preferred embodiment of the actuator assembly. One of the advantages of the actuator assembly 10 is that it permits a surgeon to apply electrical current with the forceps with the hand that is holding the forceps in the conventional manner to which the surgeon is accustomed. FIG. 9 shows that in this position the base of the users' finger FF is close to the internal pivot arm 240, which can result in unintended movement of the pivot arm and application of electric current while the surgeon is manipulating the forceps. However, the projecting hood 222 acts as a guard that prevents the user's hand from inadvertently moving the pivot arm 224 as the forceps is manipulated by the user.

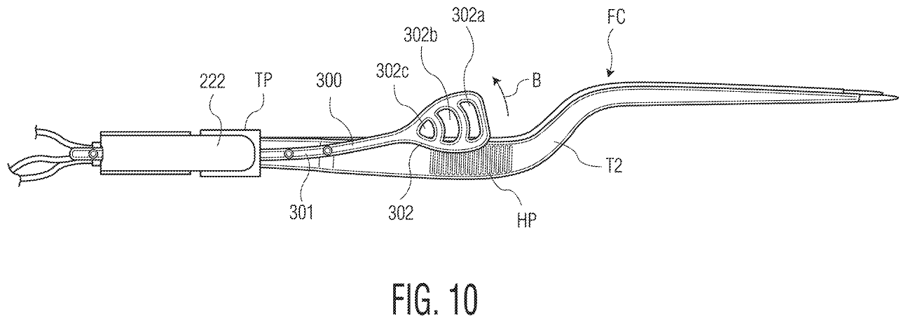

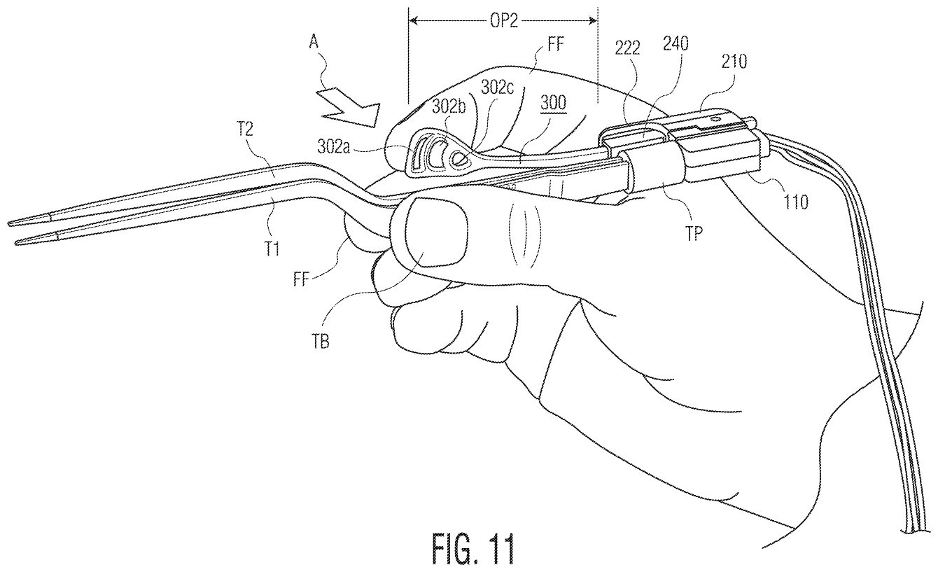

A second exemplary mode of operation will be described by reference to FIGS. 10 and 11. FIG. 10 shows the lever arm 300 bent in the plane of the drawing in the direction of the arrow B so that it will be “above” the handle portion of the forceps in the view of a user, as in FIG. 11. In this configuration the user can grip the forceps' handle portions between the thumb TB and middle finger MF, and the enlarged end of the lever arm will be located at the tip of the user's first finger FF. Thus, the user can actuate the switch actuator 112 by moving the lever arm in the direction of arrow A to rotate the pivot arm about pivot pin 242. The distance OP2 by which the lever arm 300 extends from the pivot arm can be adjusted to a length that accommodates the size of the user's hand. The contoured surface of the enlarged portion provided by the cutouts 302a, 302b, and 302c enable the user to keep his or finger properly in place for operation of the lever arm during a procedure. FIG. 11 also illustrates that the projecting hood 222 serves to reduce or eliminate the incidence of inadvertent application of electrical current. in this mode of operation.

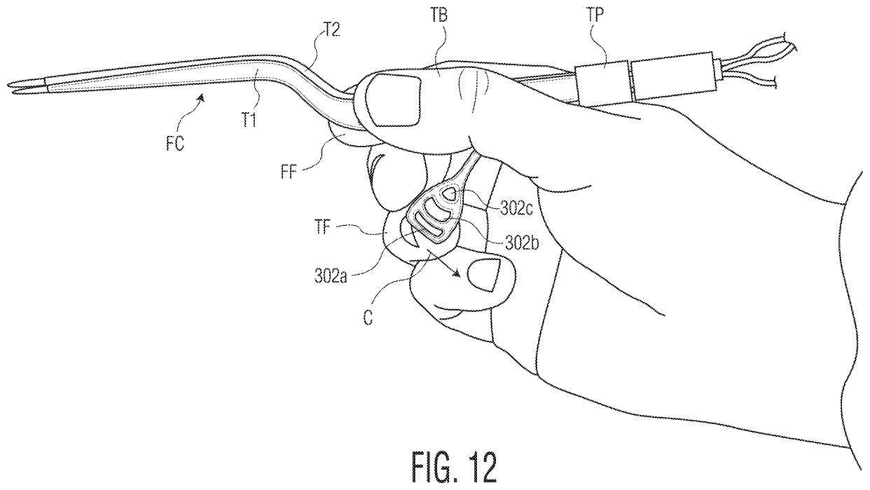

A third exemplary mode of operation is depicted in FIG. 12. In this example the lever arm 300 is bent “down” in the view of the user in the direction of the arrow C, so that when the user grasps the forceps FC between the thumb B and first finger FF, the enlarged end of the lever arm 300 will be located just at the tip of the user's third finger TF. The push-button switch actuator 112 is actuated by moving the lever arm in a direction out of the plane of the drawing (toward the viewer). In this embodiment the contoured surface of the enlarged portion (the cutouts 302a, 302b, and 302c) is an important feature because the end of the lever arm typically will not be visible to the surgeon because it is below the forceps in the normal orientation of the forceps during a procedure.

In all modes of operation the user has the option of using the actuator assembly or the foot pedal FP to introduce current to the electrodes at any time during a procedure. The user can also remove the lever arm for certain parts of a procedure and just use the foot pedal. Or the plug mount 110 with its unitary power cord 114 can be used as a conventional power cord by sliding the actuator body 200 off of the plug mount. In another embodiment the switch body 100 with the power cord 114 and the actuator body 200 comprise a unitary subassembly. This subassembly can be directly substituted for a conventional power cord and used without the lever arm in situations where the surgeon believes the lever arm could interfere with a planned procedure. In this configuration one or more lever arms can be provided separately and used as desired by inserting a lever arm into the passage 250 in the internal pivot arm 252. In another variation the entire three-component actuator assembly can be provided as a unitary structure for use as described herein without the necessity of handling multiple individual components.

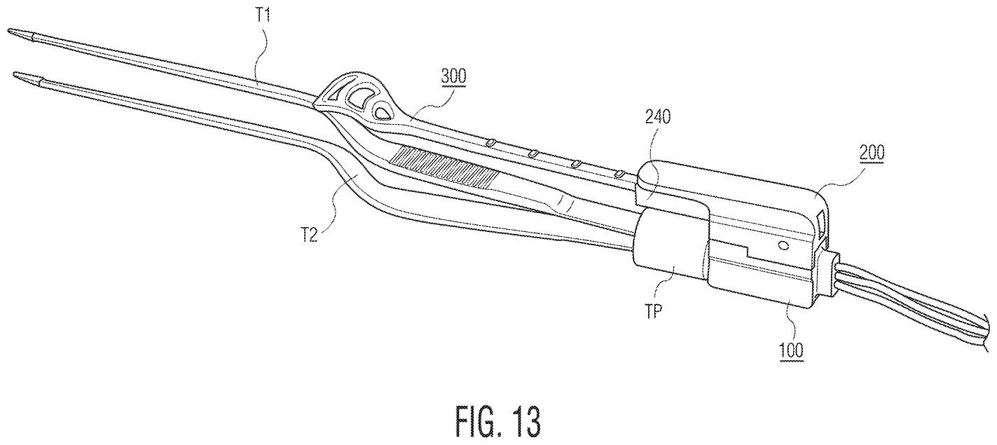

Although the above figures illustrate the actuator assembly 10 arranged for right-handed operation, another feature that further increases its versatility is the simple way in which it can be converted for left-handed operation, as shown in FIG. 13. All of the components in FIG. 13 are identical to those described above. The actuator assembly is converted to left-hand operation by rotating it 180° and plugging the mating prongs P1 and P2 on the tool plug TP into the respective sockets 110b and 100a, as discussed above in connection with FIGS. 3 and 4, thus orienting the actuator assembly so that is on the same side of the forceps as the left tine T1. The plug mount 110 and the actuator body 200 are constructed so that they are symmetrical about a plane perpendicular to a line connecting the prongs P1 and P2 of the tool plug regardless of whether they are at the left-hand or right-hand side of the forceps. Because the actuator lever arm 300 can be bent into any desired shape, the actuator assembly a left-handed user can place in position for any desired mode of operation to the same extent as a right-handed user (see above discussion in connection with FIGS. 9-12).

In an alternate embodiment the switch body 100 and the forceps comprise an integral unit. In one exemplary construction the forceps' tool plug TP and the mating sockets 110a and 110b on the switch body are replaced by an integrated structure in which the forceps' tines are directly connected to the switch body/power cord assembly to form a forceps/switch/power cord unit. The forceps can thus be connected directly to the electrical generating apparatus. In a preferred configuration, the switch body 100 is otherwise unchanged, and cooperates with the actuator body 200 and the actuator arm 300 as described above. This permits the forceps/switch/power cord unit to be used as a conventional forceps without the actuator body or the lever arm in place, or with the actuator body mounted on the switch body to enable operation in accordance with the description above.

It is anticipated that the forceps/switch/power cord unit can be manufactured a sufficiently low cost so that it can be discarded after a single use, thus avoiding potential sterilization issues presented by the switch body due to its internal circuitry and switching mechanism. The actuator body and lever arm are relatively simple in configuration and can be made without areas that present sterilization challenges. Actuator body/lever arm assemblies can be maintained in inventory for repeated use with each new disposable forceps/switch/power cord unit. Right- and left-hand versions of the disposable forceps can be made so that each has a configuration that provides the same orientation as the respective right- and left hand orientations described above and depicted in FIGS. 1 and 13. In an alternate approach, the switch body on the disposable units can have actuators (112) and connecting structure (grooves 130a and 130b) and on the left and right sides (as seen in FIG. 4) of the switch body to permit right- and left-hand operation depending on which side of the switch body the actuator housing is mounted.

### SUMMARY

The numerous constructional and operational features and advantages of the actuator assembly described herein will be immediately apparent to those skilled in the art from the above description. Those skilled in the art will readily recognize that only selected preferred embodiments of the invention have been depicted and described, and it will be understood that various changes and modifications can be made other than those specifically mentioned above without departing from the spirit and scope of the invention, which is defined solely by the claims that follow.

What is claimed is:

1. An actuator assembly adapted for use with a bipolar electrosurgical tool comprising a forceps having a handle portion at a proximal region for operation of the forceps by a hand of a user and extending generally between a tool plug at the proximal region and at least two electrodes at a distal region operatively connected to the tool plug for applying to tissue electrical current introduced to the tool plug, wherein the actuator assembly comprises:
a switch body including (i) a plug mount for removably securing the switch body to the tool plug and permitting separation of the tool plug and the switch body, (ii) a switch movable between an open position and a closed position, and (iii) a power cord for placing the switch in electrical contact with an electrical generating apparatus to introduce electrical current to the tool plug when the switch body is secured to the tool plug and the switch is in the closed position;
an actuator body comprising an actuator housing and a switch actuating member comprising a unitary structure with the switch actuating member mounted for movement relative to the actuator housing; and
an actuator lever arm mounted to the switch actuating member, wherein:
the switch body and the actuator body include respective connecting structure for removably connecting the actuator housing to the switch body, and
the switch body and actuator body are configured so that when the tool is held in the user's hand with the switch body secured to the tool plug and the actuator housing connected to the switch body, the actuator lever arm is positioned for movement by a finger of the user's hand to move the switch actuating member and place the switch in the closed position.

2. An actuator assembly as in claim 1, wherein the actuator lever arm is removably mounted to the switch actuating member.

3. An actuator assembly as in claim 1, wherein:
the actuator lever arm has a distal region spaced from the actuator housing and positioned proximate to the tool handle portion when the switch body is secured to the tool plug and the actuator housing is connected to the switch body; and
the switch actuating member is pivotally mounted to the actuator housing for rotation about a hinge point and the actuator lever arm is pivoted toward the proximate tool handle portion when moved by the user's finger.

4. An actuator assembly as in claim 3, wherein:
the actuator lever arm extends toward the distal end of the forceps generally in the direction of a longitudinal axis thereof when the switch body is secured to the tool plug and the actuator housing is connected to the switch body; and
the actuator lever arm is movably mounted to the switch actuating member for adjusting the distance between the distal region of the actuator lever arm and the actuator housing.

5. An actuator assembly as in claim 4, wherein the distal region of the actuator lever arm includes an enlarged portion for contact by the user's finger to pivot the actuator lever arm.

6. An actuator assembly as in claim 5, wherein the enlarged portion of the actuator lever arm comprises a surface curved convex-outward relative to the forceps when the switch body is secured to the tool plug for contact by the finger of the user holding the forceps for operation.

7. An actuator assembly as in claim 6, wherein the surface of the lever arm enlarged portion includes contours for providing a tactile sensation to the user's finger.

8. An actuator assembly as in claim 7, wherein the contours include depressions in the surface of the enlarged portion.

9. An actuator assembly as in claim 8, wherein the depressions are formed by openings formed through the enlarged portion.

10. An actuator assembly as in claim 9, wherein the actuator lever arm comprises a body molded around a deformable stainless steel core and includes a shaft portion mounted to the switch actuating member.

11. An actuator assembly as in claim 4, wherein:
the switch actuating member comprises a molded internal pivot arm extending from the hinge point generally in the direction of the longitudinal axis of the forceps and includes a passage for accepting the actuator lever arm to position the distal region of the actuator lever arm a desired longitudinal distance from the actuator housing; and
the internal pivot arm and the actuator lever arm include cooperating positioning means for releasably holding the actuator lever arm in a plurality of positions relative to the internal pivot arm.

12. An actuator assembly as in claim 11, wherein the distal region of the actuator lever arm includes an enlarged portion for contact by the user's finger to pivot the actuator lever arm.

13. An actuator assembly as in claim 12, wherein the positioning means includes one of (i) a plurality of detent protrusions on one of the actuator lever arm and internal pivot arm and a plurality of detent receptacles on the other of the actuator lever arm and internal pivot arm for accepting the detent protrusions, or (ii) mating screw threads on a shaft portion of the actuator lever arm and the passage in the internal pivot arm.

14. An actuator assembly as in claim 3, wherein the actuator body includes a guard positioned in relation to the actuator lever arm for protection from inadvertent movement by the user's hand.

15. An actuator assembly as in claim 1, wherein the actuator lever arm comprises a body molded around a deformable stainless steel core.

16. An actuator assembly as in claim 1, wherein the connecting structure includes at least one groove on one of the actuator housing and plug mount and at least one ridge on the other of the actuator housing and plug mount, and wherein the at least one groove slidingly accepts the at least one ridge to removably connect the actuator body to the switch body.

17. An actuator assembly adapted for use with a bipolar electrosurgical tool comprising a forceps having a handle portion at a proximal region for operation of the forceps by a hand of a user and extending generally between a tool plug at the proximal region and at least two electrodes at a distal region operatively connected to the tool plug for applying to tissue electrical current introduced to the tool plug, wherein the actuator assembly comprises:
a switch body including (i) a plug mount for removably securing the switch body to the tool plug and permitting separation of the tool plug and the switch body, (ii) a switch movable between an open position and a closed position, and (iii) a power cord for placing the switch in electrical contact with an electrical generating apparatus to introduce electrical current to the tool plug when the switch body is secured to the tool plug and the switch is in the closed position; and
an actuator body comprising an actuator housing and a switch actuating member mounted for movement relative to the actuator housing, wherein:
the switch body and the actuator body comprise a unitary structure,
the switch actuating member removably accepts an actuator lever arm, and
the switch body and actuator body are configured so that when the tool is held in the user's hand with the switch body secured to the tool plug, the actuator lever arm is positioned for movement by a finger of the user's hand to move the switch actuating member and place the switch in the closed position.

18. An actuator assembly as in claim 17, wherein:
the actuator lever arm has a distal region spaced from the actuator housing and positioned proximate to the tool handle portion when the switch body is secured to the tool plug and the actuator housing is connected to the switch body; and
the switch actuating member is pivotally mounted to the actuator housing for rotation about a hinge point and the actuator lever arm is pivoted toward the proximate tool handle portion when moved by the user's finger.

19. An actuator assembly as in claim 17, wherein:
the switch actuating member comprises a molded internal pivot arm extending from the hinge point generally in the direction of a longitudinal axis of the forceps and includes a passage for accepting the actuator lever arm to position the distal region of the actuator lever arm a desired longitudinal distance from the actuator housing; and
the internal pivot arm and the actuator lever arm include cooperating positioning means for releasably holding the actuator lever arm in a plurality of positions relative to the internal pivot arm.

20. An actuator assembly as in claim 19, wherein the distal region of the actuator lever arm includes an enlarged portion for contact by the user's finger to pivot the actuator lever arm.

21. An actuator assembly as in claim 20, wherein the positioning, means includes one of (i) a plurality of detent protrusions on one of the actuator lever arm and internal pivot arm and a plurality of detent receptacles on the other of the actuator lever arm and internal pivot arm for accepting the detent protrusions, or (ii) mating screw threads on a shaft portion of the actuator lever arm and the passage in the internal pivot arm.

22. An actuator assembly as in claim 17, wherein the actuator body includes a guard positioned in relation to the actuator lever arm for protection from inadvertent movement by the user's hand.

23. A power cord assembly including a switch body adapted for use with a bipolar electrosurgical tool comprising a forceps having a handle portion at a proximal region for operation of the forceps by a hand of a user and a longitudinal axis extending generally between a tool plug at the proximal region and at least two electrodes at a distal region operatively connected to the tool plug for applying to tissue electrical current introduced to the tool plug, wherein the switch body comprises:
a plug mount for removably securing the switch body to the tool plug and permitting separation of the tool plug and the switch body;
a switch movable between an open position and a closed position;
a power cord for placing the switch in electrical contact with an electrical generating apparatus to introduce electrical current to the tool plug when the switch body is secured to the tool plug and the switch is in the closed position; and
connecting structure for removably connecting the switch body to an actuator body having a switch actuating member movable relative to the switch body when the actuator body is connected to the switch body to close the switch when the switch actuating member is moved by a user of the forceps.

24. A power cord assembly as in claim 23, wherein the power cord includes power leads for connecting to power terminals of an electrical generating apparatus and a control lead for connecting to a control input of the electrical generating apparatus for introducing electrical current to the tool plug when the switch body is secured to the tool plug and the switch is closed.

25. An actuator lever arm adapted for use with a bipolar electrosurgical tool comprising a (i) forceps having a handle portion at a proximal region for operation of the forceps by a hand of a user and extending generally between a tool plug at the proximal region and at least two electrodes at a distal region operatively connected to the tool plug for applying to tissue electrical current introduced to the tool plug, and (ii) an actuator assembly mounted to the forceps and including a switch movable between an open position and a closed position and a switch actuating member for closing the switch and introducing electrical current to the electrodes, the actuator lever arm comprising:
a body molded around a deformable stainless steel core;
a shaft portion for mounting the actuator lever arm to the switch actuating member; and
an enlarged portion for contact by the user's finger to pivot the actuator lever arm and close the switch when the actuator lever arm is moved by the user.

26. An actuator lever arm as in claim 25, wherein:
the actuator lever arm extends toward the distal end of the forceps generally in the direction of a longitudinal axis thereof when mounted to the actuator assembly; and
the actuator lever arm is movably mounted to the switch actuating member for adjusting the distance between the distal region of the actuator lever arm and the actuator assembly.

27. An actuator lever arm as in claim 25, wherein the enlarged portion of the actuator lever arm comprises a surface curved convex-outward relative to the forceps when the lever arm is mounted to the switch actuating member.

28. An actuator lever arm as in claim 25, wherein the surface of the lever arm enlarged portion includes contours for providing a tactile sensation to the user's finger.

29. An actuator lever arm as in claim 28, wherein the contours include depressions in the surface of the enlarged portion.

30. An actuator lever arm as in claim 29, wherein the depressions are formed by openings formed through the enlarged portion.

31. An actuator lever arm as in claim 25, wherein the shaft portion includes positioning means for cooperating with corresponding positioning means on the switch actuating member, the positions means comprising one of (i) a plurality of detent protrusions on, one of the actuator lever arm and the switch actuating member and a plurality of detent receptacles on the other of the actuator lever arm and switch actuating member for accepting the detent protrusions, or (ii) mating screw threads on the shaft portion of the actuator lever arm and the switch actuating member.

32. A bipolar electrosurgical tool comprising:
a forceps including two tines extending from a proximal end to a distal end, wherein each tine has a handle portion between the distal and proximal ends and an electrode at the distal end for applying to tissue electrical current introduced to the times at the proximal end thereof;
a switch body mounted to the distal end of the forceps and including (i) a switch movable between an open position and a closed position, and (ii) connecting structure for removably connecting to the switch body a switch actuating member for operation by a hand of a user holding the handle portions of the tines to place the switch in the closed position; and
a power cord mounted to the switch body for placing the switch in electrical contact with an electrical generating apparatus to introduce electrical current to the tines when the switch is in the closed position.

33. A bipolar electrosurgical tool as in claim 32, wherein said switch actuating member accepts an actuator lever arm for movement by the hand of the user to operate the switch actuating member.

34. A bipolar electrosurgical tool as in claim 33, wherein said switch body includes right- and left-hand connecting structure for selectively orienting the actuator lever arm relative to the handle portions for operation by the right or left hand of the user.
